(19) United States
(12) Patent Application Publication
(10) Pub. No.: US 2017/0100034 A1
(43) Pub. Date: Apr. 13, 2017

(54) SYSTEM AND METHOD FOR REMOTELY MONITORING ELECTROMYOGRAPHY THERAPY

(71) Applicant: Dynofit, Inc., Los Alamos, NM (US)

(72) Inventors: Rohan C. Loveland, Carrollton, TX (US); Maria E. Loveland Schneider, Carrollton, TX (US)

(21) Appl. No.: 15/288,919

(22) Filed: Oct. 7, 2016

Related U.S. Application Data
(60) Provisional application No. 62/239,153, filed on Oct. 8, 2015.

(51) Int. Cl. A61B 5/00; A61B 5/11; A61B 5/0492

(52) U.S. Cl. CPC A61B 5/0004; A61B 5/0022; A61B 5/0492; A61B 5/1107; A61B 5/4833; A61B 5/6833; A61B 5/742; A61B 2505/09; A61B 2562/0209

(57) ABSTRACT

Systems and methods for remotely monitoring electromyography therapy include receiving an analog signal from an electromyography sensor associated with contraction of a muscle of a first patient. The analog signal is converted to a digital signal. The digital signal is communicated wirelessly to a remote computing device using a communications network. The digital signal is converted into human understandable data by the remote computing device, and it is displayed graphically by the remote computing device.

### PRIORITY CLAIM

This application claims priority from U.S. Provisional Patent Application Ser. No. 62/239,153 filed on Oct. 8, 2015, which is hereby incorporated by reference.

### Cross-Reference to Related Applications

This application is related to U.S. patent application Ser. No. 14/848,794 filed on Sep. 9, 2015 and entitled “GAMIFIED ELECTROMYOGRAPHIC NEUROMUSCULAR REEDUCATION THERAPY SYSTEM,” which claims priority to U.S. Provisional Patent Application Ser. No. 62/047,658, filed Sep. 9, 2014, each of which is hereby incorporated by reference.

### STATEMENT REGARDING FEDERALLY SPONSORED RESEARCH OR DEVELOPMENT

This invention was made in part with government support under 1R43HD080234-01 awarded by the National Institute of Health. The government has certain rights in the invention.

### BACKGROUND OF THE DISCLOSURE

Eight million people in the U.S. suffer from strokes, cerebral palsy, and spinal cord injuries—all of which can result in a patient's loss of the ability to properly control his/her muscles. A variety of other neurological conditions, musculoskeletal injury, and surgeries can also result in loss of or reduced muscle control.

Motor skill issues can be addressed through neuro-muscular re-education therapies (NMRT) to dramatically improve a person's quality of life (e.g. a person might re-learn to walk). The currently available technology for performing electromyography (EMG) based NMRT usually connects a dedicated computer to the EMG sensors placed on a single patient either wirelessly or with wires. Thus, EMG monitoring is typically done in a clinical setting under the direct supervision of a physical therapist or occupational therapist. Often the therapist will instruct the patient to perform certain exercises as part of the physical therapy at home upon discharge. The therapist then relies on self-reporting by the patient at a subsequent therapy session as to whether the therapy protocol was strictly followed by the patient.

### SUMMARY

Embodiments of the present disclosure include systems and methods for remotely monitoring electromyography therapy. The method includes receiving an analog signal from an electromyography sensor associated with contraction of a muscle of a first patient. The analog signal is converted to a digital signal. The digital signal is communicated wirelessly to a remote computing device using a communications network. The digital signal is converted into human understandable data by the remote computing device, and it is displayed graphically by the remote computing device.

According to certain embodiments, the remote computing device receives signals indicating the contraction of the muscles of multiple patients and it is operable to display on a single screen data indicating the therapy adherence of each patient.

An additional embodiment is a computer implemented system for remotely monitoring electromyography therapy. The system includes a first electromyography sensor configured to be coupled to a first patient that is operable to wirelessly transmit a signal associated with contraction of a muscle of the first patient. A second electromyography sensor is configured to be coupled to a second patient and is operable to transmit a signal associated with contraction of a muscle of the second patient. A software program, when executed on a computing device remote from the first and second patient, is operable to convert the signals associated with the first patient and the signals associated with the second patient into human understandable data. The software program is further operable to display the human understandable data associated with the first patient and the human understandable data associated with the second patient.

Technical advantages of embodiments disclosed include the elimination of the reliance on self-reporting adherence to prescribed therapy protocols. According to the teachings of the present disclosure, a single therapist can monitor the prescribe therapy of multiple patients, and thereby take action, if necessary, to ensure that patients are performing prescribe muscle therapy and that the recovery of the patient is progressing as expected.

Other technical advantages include the ability of an interested person, such as a family member, to remotely monitor the therapy and recovery progress of another family member. Thus, the family member has information from which to take action to ensure that exercises prescribed by a therapist are properly performed according to instructions of a therapist.

Other aspects, features, and advantages will become apparent from the following detailed description when taken in conjunction with the accompanying drawings, which are part of this disclosure and which illustrate, by way of example, principles of the inventions disclosed.

### BRIEF DESCRIPTION OF THE FIGURES

The following Detailed Description references the accompanying Drawings wherein:

FIG. 1A is a schematic diagram of a system for remotely monitoring electromyography therapy according to an embodiment of the present disclosure;

FIG. 1B is a schematic diagram of a remote computing device and/or a mobile device according to the teachings of the present disclosure;

FIG. 2 is a schematic diagram of the portion of the system shown in FIG. 1 pertaining to a single patient;

FIG. 3 illustrates an exemplary EMG sensor for use with the system for remotely monitoring electromyography therapy shown in FIG. 2;

FIG. 4 is a schematic diagram of the components of the exemplary EMG sensor shown in FIG. 3;

FIG. 5 is a screen shot of a display of an EMG therapy aggregator software program used in implementing the system for remotely monitoring EMG therapy according to the teachings of the present disclosure;

FIG. 6 is a screen shot of a screen different from the screen shown in FIG. 5 of the EMG aggregator software program used in implementing the system for remotely monitoring EMG therapy according to the teachings of the present disclosure; and

FIG. 7 is a flow diagram of a method for remotely monitoring EMG therapy according to the teachings of the present disclosure.

### DETAILED DESCRIPTION

Embodiments described herein generally relate to a system and method for remotely monitoring neuro-muscular re-education therapy using electromyography sensors and one or more remote computing devices. Motor skill issues can be addressed through neuro-muscular re-education therapies (NMRT) to dramatically improve a person's quality of life (e.g. a person might re-learn to walk).

The embodiments described herein provide a system and method for a therapist to remotely monitor the therapeutic exercises and activities prescribed to a plurality of patients. Although this disclosure uses the term “therapist” for simplicity, it is understood that a therapist could be any individual that has an interest in the muscular therapy of an individual. For example, a coach or trainer that monitors the muscular therapy and recovery of an athlete may be considered a therapist for purposes of this disclosure. Likewise, a family member, such as a husband, wife, son, daughter, and the like who may be interested in ensuring that a loved one is performing prescribed muscular therapy and is recovering as expected may also be considered a therapist or purposes of this disclosure. The system and method of the present disclosure facilitates remote monitoring of a patient's adherence to a therapy protocol and progress while the patient is outside the clinical setting and typically performing muscle strengthening exercises at home or at a gym without direct “therapist” involvement. Moreover, the scope of remotely monitoring the therapy of the patient includes physical, occupational and other therapies, as well as athletic and fitness training.

The therapist receives information from an electromyography (EMG) sensor attached to the skin surface above a muscle that is to be contracted or otherwise exercised according to the instructions of the therapist. That is, the therapist may instruct the patient in the proper placement and application technique of surface EMG sensors over particular muscles in a clinical setting, and the patient can be released to home and apply the EMG sensors as taught and the data regarding contraction of the patient's muscles can be communicated to the therapist and be viewed at a computer located at the office of the therapist or a mobile device used by the particular therapist. In this manner, a therapist and a caregiving team may remotely monitor the performance of therapeutic neuromuscular exercises to ensure that the given protocol is adhered to, the muscles are strengthening as expected, and the therapist can take action if information from the EMG indicates that the prescribed therapy is being performed incorrectly or is not being performed at all. The electromyography (EMG) based NMRT is remotely monitored by the caregiving team having an interest in the recovery of the patient, which may thereby increase patient performance of prescribed NMRT and to increase therapeutic efficiency. In addition, a family member of the patient who is not a member of the clinical therapy team may also monitor the EMG therapy through his computing device or mobile communication device, such as a smart phone with an appropriate application running on the smart phone. The interface of the smart phone application may be particularly understandable by a user with limited or no medical training.

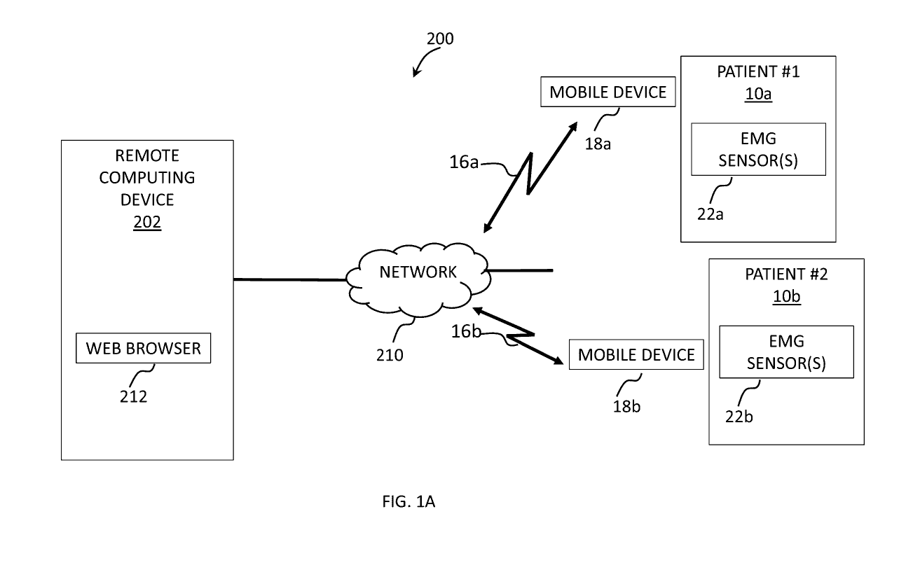

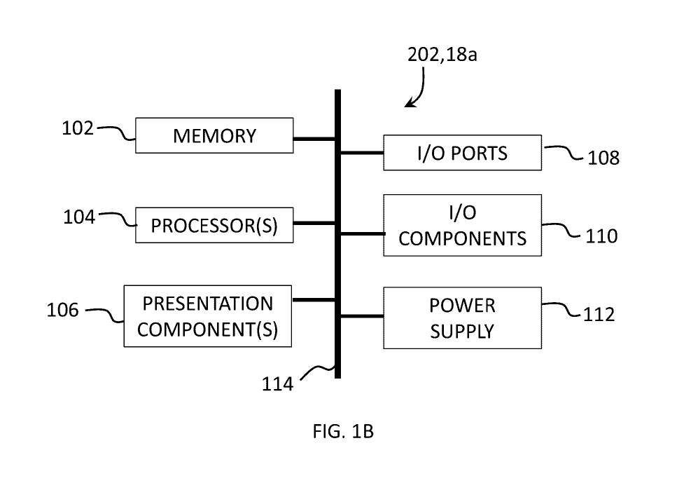

FIG. 1A is a schematic drawing of a system 200 for remotely monitoring electromyography therapy. A remote computing device 202 is operated by a therapist or other interested party. The remote computing device communicates through the communications network 210, such as the Internet, through either wired or wireless communication. For example, the remote computing device 202 may run a web-based program or application through a web browser 212.

An EMG sensor 22a is attached to the skin of a patient 10a. A second EMG sensor 22b is attached to the skin of a second patient 10b. Any number of patients may be remotely monitored by the system 200. The patient 10a and the patient 10b may be unrelated to each other with the exception that they are both receiving muscular therapy from the same therapist or the same therapy clinic such that data from each EMG sensor 22a and 22b is communicated through the network 210 and received by the remote computing device 202 where such data remains associated with the respective patient and is viewable by a therapist. According to the teaching of the present disclosure, the therapist may take action based on the data from a respective EMG sensor 22a or 22b to ensure compliance to a particular muscular therapy protocol and may also monitor the progress of the therapy on the targeted muscles or muscle group.

According to some embodiments, the EMG sensor 22a transmits a signal to a mobile device 18a. The signal may be a wireless signal, such as a radio signal, and the mobile device may be a smartphone or a tablet computer. According to one embodiment, the mobile device is a personal smartphone or tablet computer of the patient 10a that is operable to run a mobile application that allows the smart phone to receive the signals from the EMG sensor 22a. According to an alternate embodiment, the EMG sensor may communicate with the mobile device 18a through wired communication.

The mobile device 18a processes the data from the EMG sensor 22a and transmits a signal to the network 210 where it may ultimately be received by the remote computing device 202. The data from the EMG sensor 22a may be stored either temporarily or permanently on the mobile device 18a. Alternatively or additionally, the data may be communicated to a cloud server where it may be stored for future access by either the mobile device 18a or the remote computing device 202. Although FIG. 1A only illustrates two patients 10a and 10b having a single EMG sensor each, this disclosure contemplates numerous patients 10a-10n where each patient may have multiple EMG sensors 22aa-22an positioned to receive electrical signals from multiple muscles or muscle groups.

Reference is now made to FIG. 1B, which illustrates the basic components of each of the remote computing device 202 and the mobile device 18a, 18b. The remote computing device 202 and/or the mobile device 18a is but one example of a suitable computing environment and is not intended to suggest any limitations as to the scope of use or functionality of the disclosure. Neither should computing device 202 or mobile device 18a be interpreted as necessarily having any dependency or requirement relating to any one or combination of components illustrated. The disclosure may be described in the general context of a computer code or machine-usable instructions, including computer-executable instructions such as program modules, being executed by a computer or other machine, such as a PDA, smartphone or other handheld device. Generally, program modules that include routines, programs, objects, components, smart-phone applications, data structures, and the like, refer to code that perform particular tasks or implement particular abstract data types. The modules described herein may represent executable source code written in a well-known language, such as, for example, C, C++, C#, Java, or the like. Embodiments described herein may be practiced in distributed computing environments where tasks are performed by remote-processing devices that are linked through one or more communications networks.

With continued reference to FIG. 1B, remote computing device 202 and/or mobile device 18a, 18b, includes a bus 114 that directly or indirectly couples the following devices: memory 102, one or more processors 104, one or more presentation components 106, input/output ports 108, input/output components 110 and an illustrative power supply 112. Bus 114 represents what may be one or more busses (such as an address bus, data bus, or combination thereof). Although the various blocks of FIG. 1B are shown with lines for the sake of clarity, in reality, delineating various components is not so clear, and metaphorically, the lines would more accurately be grey and fuzzy. For example, one may consider a presentation component such as a display device to be an I/O component. Also, processors have memory. It will be understood by those skilled in the art that such is the nature of the art, and, as previously mentioned, the diagram of FIG. 1B is merely illustrative of an exemplary computing device that can be used in connection with one or more embodiments of the present invention. Distinction is not made between such categories as “workstation,” “server,” “laptop,” “hand-held device,” “smartphone,” etc., as all are contemplated within the scope of FIG. 1B and reference to as a “computing device” or a “mobile device.”

Each of the remote computing device 202 and/or the mobile device 18a, 18b typically includes a variety of computer-readable media. By way of example, and not limitation, computer-readable media may comprise Random Access Memory (RAM); Read Only Memory (ROM); Electronically Erasable Programmable Read Only Memory (EEPROM); flash memory or other memory technologies; CDROM, digital versatile disks (DVD) or other optical or holographic media; magnetic cassettes, magnetic tape, magnetic disk storage or other magnetic storage devices, carrier wave or any other medium that can be used to encode desired information and be accessed by remote computing device 202 and/or mobile device 18a, 18b.

The memory 102 includes computer-storage media in the form of volatile and/or nonvolatile memory. The memory 102 may be removable, nonremovable, or a combination thereof. Exemplary hardware devices include solid-state memory, hard drives, cache, optical-disc drives, etc. The remote computing device 202 and/or mobile device 18a, 18b, each includes one or more processors 104 that read data from various entities such as memory 102 or I/O components 110. The presentation component(s) 106 present data indications to a user or other device. Exemplary presentation components 106 include a display device, speaker, printing component, vibrating component, etc.

The I/O ports 110 allow computing device 202 to be logically coupled to other devices including I/O components 110, some of which may be built in. Illustrative components include a microphone, EMG sensors, wireless transceivers, joystick, game pad, satellite dish, scanner, printer, wireless device, keypad etc.

Returning to FIG. 1A, the network 210 may include any computer network or combination thereof. Examples of computer networks configurable to operate as network 210 include, without limitation, a wireless network, landline, cable line, digital subscriber line (DSL), fiber-optic line, local area network (LAN), wide area network (WAN), metropolitan area network (MAN), Bluetooth connection, or the like. The network 210 is not limited, however, to connections coupling separate computer units. Rather, the network 210 may also include subsystems that transfer data between servers or computing devices. For example, the network 210 may also include a point-to-point connection, the Internet, an Ethernet, an electrical bus, a neural network, or other internal system.

In an embodiment where the network 210 comprises a LAN networking environment, components may be connected to the LAN through a network interface or adapter. In an embodiment where the network 210 comprises a WAN networking environment, components may use a modem, or other means for establishing communications over the WAN, to communicate. In embodiments where the network 210 comprises a MAN networking environment, components may be connected to the MAN using wireless interfaces or optical fiber connections. Such networking environments are commonplace in offices, enterprise-wide computer networks, intranets, and the Internet. It will be appreciated that the network connections shown are exemplary and other means of establishing a communications link between the computers may also be used.

Furthermore, the network 210 may also include various components necessary to facilitate communication with a mobile phone (e.g., cellular phone, Smartphone, Blackberry®). Such components may include, without limitation, switching stations, cell sites, Public Switched Telephone Network interconnections, hybrid fiber coaxial cables, or the like.

In some embodiments, remote computing device 202 and/or the mobile device 18a, 18b each includes a web browser 212 which is a software application enabling a user to display and interact with information located on a web page. The web browser 212 may locate web pages by sending a transferring protocol and the URL. The web browser 212 may use various URL types and protocols, such as hypertext transfer protocol (HTTP), file transfer protocol (FTP), real-time streaming protocol (RTSP), etc. The web browsers 212 may also understand a number of file formats—such as HTML, graphics interchange format (GIF), tagged image file format (TIFF), portable document format (PDF), or joint photographic experts group (PDF) file format, and the like—the wealth of which can be extended by downloaded plug-ins. Additionally, the web browser 212 may be any browser capable of navigating the Web, such as Internet Explorer®, Netscape Navigator, Mozilla, Firefox, etc.

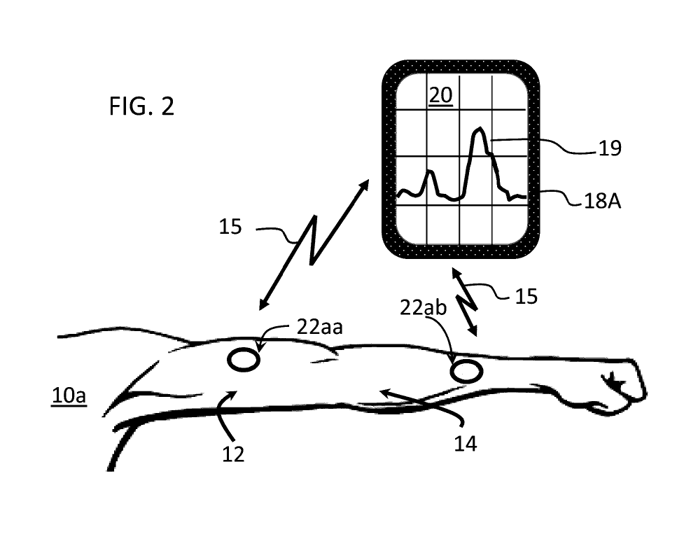

FIG. 2 schematically illustrates an environment where EMG sensors 22aa and 22ab are used by the patient 10a. The EMG sensor 22aa is secured to the skin above the bicep muscle, and the EMG sensor 22ab is secured to the skin of the patient 10a above a forearm muscle.

An electric potential is detected or otherwise received and recorded by the EMG sensor 22aa when the patient 10a voluntarily contracts his bicep muscle in accordance with a particular neuro-muscular therapy protocol, which may have been previously taught to the patient 10a by a physical therapist. The signal associated with the electrical potential created by the contraction of the bicep muscle is processed by the EMG sensor 22aa as described in more detail below. Then a digital signal is transmitted by wireless radio signal 15 from the EMG sensor 22aa to the mobile device 18a, which is illustrated as a tablet computer. The mobile device processes the received wireless radio signal 15 and represents the data associated with the signal 15 graphically indicated by wave graph 19, which is displayed on the display screen 20 of the mobile device 18a. According to one embodiment, the wave graph 19 may be a rectified or non-rectified electromyogram. The display screen 20 may display a graphical user interface. An example display screen 20 is a touchscreen of a smartphone or a tablet computer. The wireless radio signal may be a BlueTooth signal as is known in the art.

The EMG sensor 22ab positioned on the forearm 14 of the patient operates as described above with respect to EMG sensor 22aa with the exception that the mobile device 18a associates the received radio signal 15 with contraction of a muscle of a forearm 14.

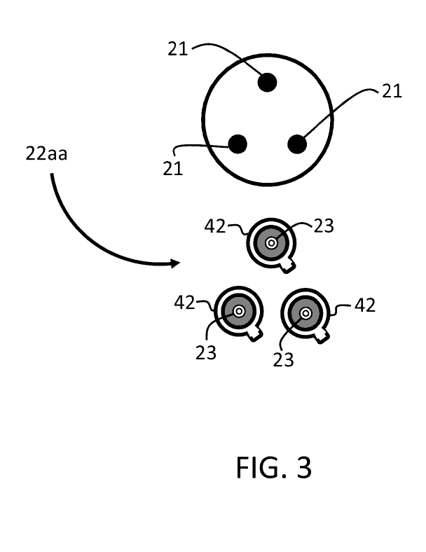

FIG. 3 illustrates the EMG sensor 22aa with electrodes separated from their receiving component. As discussed above, the patient 10a may position the EMG sensor 22aa on the skin proximate a targeted muscle or muscle group as directed by a therapist. A plurality of electrode pads 42 are adhered to the skin of the patient 10a. According to certain embodiments, the electrode pads are disposable electrode pads 42. According to an alternate embodiment, the electrode pads 42 may be reusable electrodes, rather than disposable pads 42. The function of either the disposable or reusable electrodes may be enhanced by application of a moistening or conductive gel to either the skin of the patient 10a or to the underside of the electrode 42 that is configured to be adhered to the skin of the patient 10a. An example of a wireless electromyography sensor is described by U.S. Pat. No. 6,643,541 filed on Dec. 7, 2001 and issued to Mok et al., which is hereby incorporated by reference.

A separate member of the EMG sensor 22aa includes snap fasteners 21 configured to snap to a corresponding snap fastener 23 on the disposable electrode pads 42 that are adhered to the skin of the patient 10a.

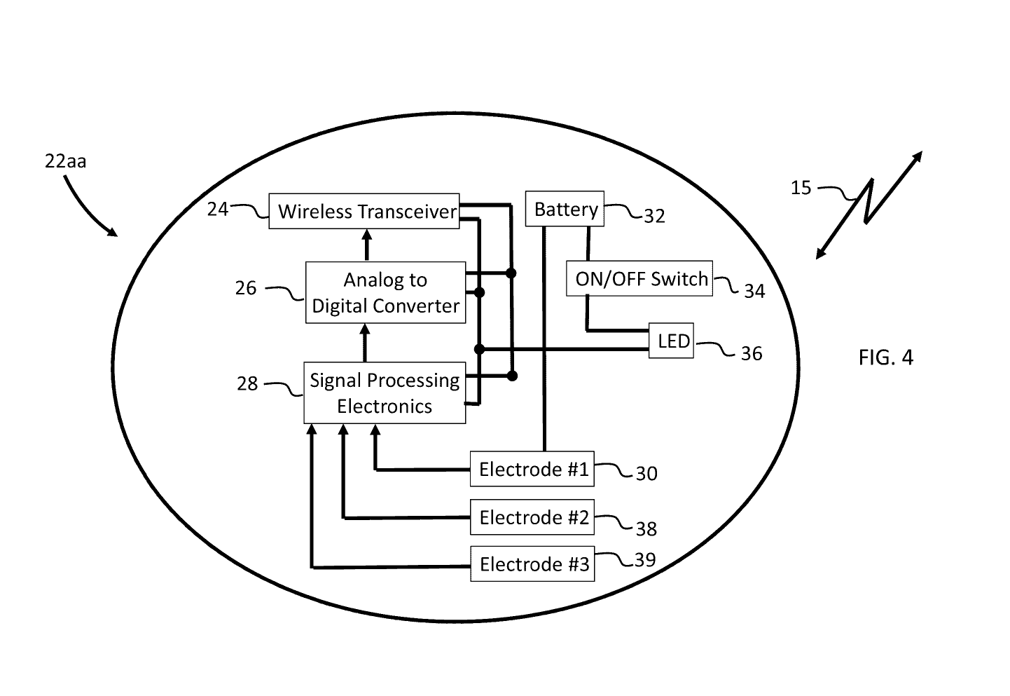

An embodiment of the components of EMG sensor 22aa is shown schematically in FIG. 4. A battery 32 powers the system and is activated using ON/OFF switch 34. In one embodiment, LED 36 illuminates when the battery 32 is delivering power such that EMG sensor 22aa is operational. According to an alternate embodiment, the EMG sensor 22aa does not include an ON/OFF switch 34. Rather, the EMG sensor 22aa may be always on and may become active and operable to detect and transmit signals associated with muscle contraction upon receiving a signal from the mobile device 18a. The signal received from the mobile device 18a indicates to the EMG sensor 22aa that the mobile device 18a is within range and operable to receive signals from the EMG sensor 22aa.

A first electrode 30, a second electrode 38, and a third electrode 39 are coupled to the skin proximate a target muscle or muscle group of the patient 10a. The voluntary contraction of the muscle, for example the bicep muscle, by the patient 10a is detected and recorded by signal processing electronics 28. The processed signal from signal processing electronics 28 is digitized by analog to digital converter 26. In some embodiments, the digitized signal is transmitted wirelessly by wireless transceiver 24 as wireless radio signal 15 to mobile device 18a (FIG. 2). Mobile device 18a then communicates a corresponding signal using the communications network 210, which is ultimately received by the remote computing device 202.

It should also be noted that wireless transceiver 24 might be based on any of a range of radio frequency technologies, such as Bluetooth or other short range wireless communication protocol, 2.4 GHz, or it might be optical or infrared. Likewise, the signal processing electronics 28 could vary widely in their actual components, as could analog to digital converter 26.

While, the illustrated embodiment of the EMG sensor 22aa shown in FIGS. 3 and 4 has three electrodes with fixed spacing, the EMG sensor 22aa may have any suitable number of electrodes in other embodiments. For example, in some embodiments the EMG sensor 22aa has two electrodes. In other embodiments, either two or three electrodes may be positioned with variable spacing. EMG sensor 22aa with fixed spaced electrodes may increase ease and simplicity in which the electrodes are placed by the patient 10a, as opposed to variable spaced electrodes where each electrode is to be precisely placed by the patient 10a.

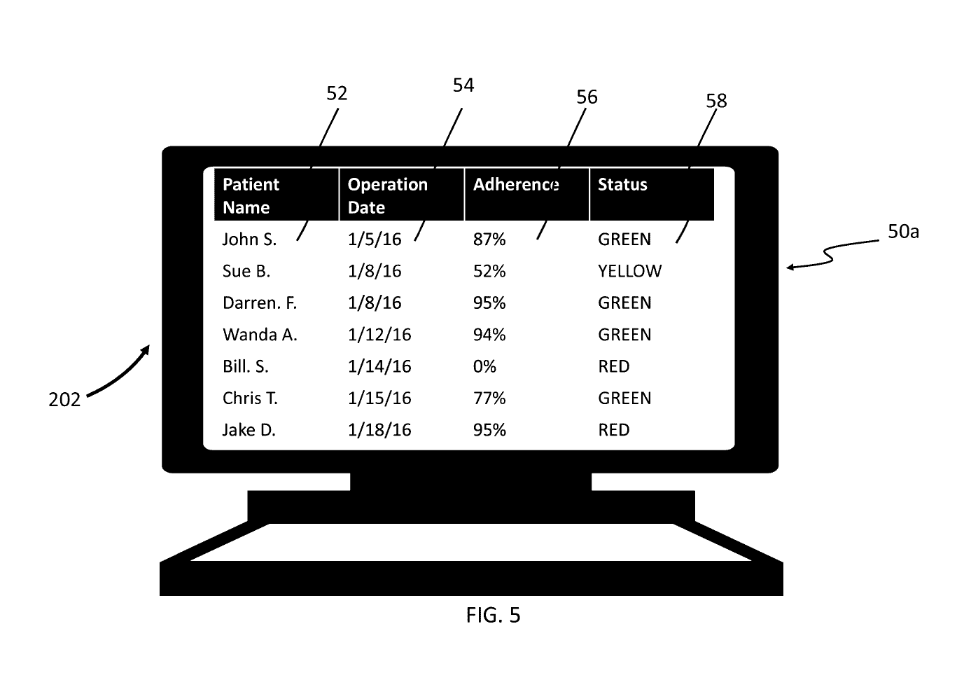

FIG. 5 illustrates a screen shot 50a of a computer device, such as computing device 202, running an EMG patient aggregator software program according to the teachings of the present disclosure. The EMG patient aggregator software program allows a therapist to remotely track and monitor the EMG therapy of a plurality of patients, for example the patients 10a-10n. Data for seven patients is shown. The display may display a multitude of information associated with a particular patient's EMG therapy. For example, the software program may retrieve information stored in a relational database where each therapy patient is assigned a unique identification number that is stored in the database. This number is associated with the particular data of a particular patient, specifically data associated with EMG sensors that the particular patient has been instructed to place on targeted muscles in connection with his or her physical therapy.

According to one embodiment, data fields stored and displayed by the patient EMG therapy aggregator program include the name of a patient 52, a date data field 54 showing which date and or time EMG signals were detected, which coincides with a date the patient performed his or her EMG therapy. The EMG aggregator program may also include a data field associated with the adherence percentage data field 56, which indicates whether the received EMG sensor signals indicate compliance with a predetermined EMG therapy protocol. For example, the program may be programmed with a certain number of repetitions of a certain muscle or group of muscle to exercise per day. The program compares the EMG sensor data associated with the signals detected by the EMG sensor with the preprogrammed number of repetitions and indicates a percentage of adherence. If the percentage of adherence is below a predetermined threshold, a status data field 58 may indicate a green, yellow, or red status, depending on how far out of adherence a particular patient is to a prescribed EMG therapy protocol. According to certain embodiments, the status field may be displayed as a highlighted color of the row associated with the particular patient. In addition, if the patient adherence drops below a predetermined threshold, the program may send an alert to an interested party, such as a therapist. The alert may be in the form of an email or a text message to a smartphone of the interested party.

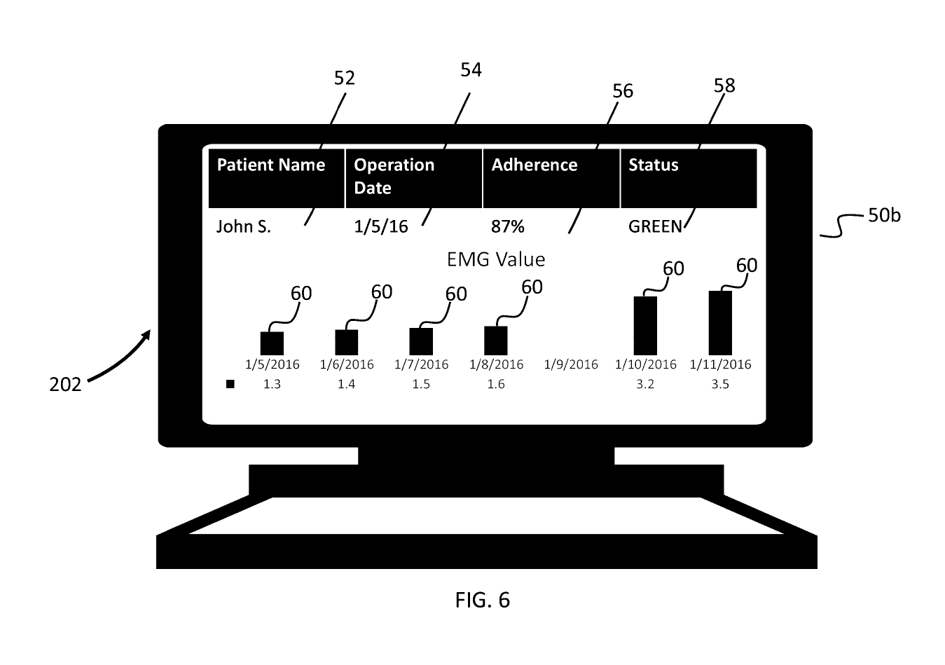

FIG. 6 is a screen shot 50b of an additional display of the EMG aggregator software program described with respect to FIG. 5. The computing device 202 may display the screen shown in FIG. 6 if a row of a particular patient is selected by the therapist by, for example clicking a mouse or tapping a touch screen. Additional information from the relational database associated with a particular patient may be displayed. For example a bar graph 60 is shown for each day the patient identified in the selected name data field 52 performed his EMG therapy. The data may be displayed in any suitable form such as a bar graph indicating a value associated with the strength of the electrical signals created by the nerves associated with the targeted muscles being exercised as part of the physical therapy protocol. Using this display, a therapist may graphically see data associated with the progress of the physical therapy performed by the patient, where the data originates at the EMG sensor applied to the patient, and in this manner the therapist may accurately monitor the EMG therapy performed by a patient from a remote computing device.

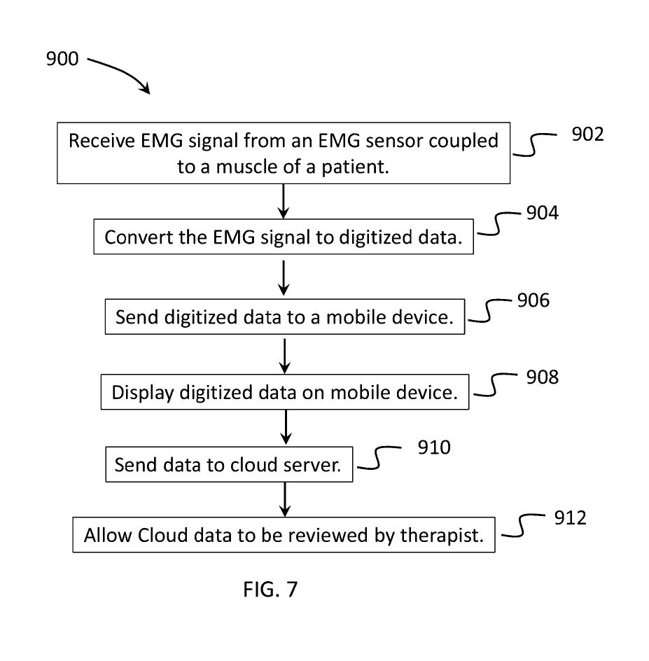

FIG. 7 is a flow diagram of a method 900 of remotely monitoring electromyography therapy that is implemented with computer-executable program(s) or computer-executable instruction(s) in computer system(s) or device(s). In some embodiments, the method 900 includes receiving an EMG signal from an EMG sensor coupled to a muscle or group of muscles of a patient, as shown at block 902. The EMG signal is converted to digitized data using an analog to digital converter, as shown at block 904. The digitized data is transmitted by a wireless transceiver that is embedded in the EMG sensor to a mobile device at step 906, such as a smartphone, a tablet computer, or a laptop computer. According to certain embodiments, the digitized data may be communicated to a desktop computer, which in this example may be considered a mobile device. The digitized data is displayed in a human-readable form on the mobile device at step 908. For example, the mobile device may display the digitized data received from the EMG sensor as an electromyogram, which may or may not be rectified to account for negative values associated with relaxation of the targeted muscle or muscle group.

At step 910, the mobile device communicates digitized data indicating the signals received and recorded by the EMG sensor to the cloud through a communications network, such as the Internet. The cloud is generally one or more data storage servers accessible through the communications network by a user with appropriate permissions, such as a pre-assigned or pre-generated and recognized username and password. A therapist or other caregiver or coach may receive the digitized data either from the cloud or more directly through the communications network from the mobile device at step 912. According to some embodiments, the remote computing device operates a web browser that is configured to display data associated with multiple EMG sensors receiving signals from multiple patients as shown and described with respect to FIGS. 5-6.

In the foregoing description of certain embodiments, specific terminology has been resorted to for the sake of clarity. However, the disclosure is not intended to be limited to the specific terms so selected, and it is to be understood that each specific term includes other technical equivalents which operate in a similar manner to accomplish a similar technical purpose.

In this specification, the word “comprising” is to be understood in its “open” sense, that is, in the sense of “including”, and thus not limited to its “closed” sense, that is the sense of “consisting only of”. A corresponding meaning is to be attributed to the corresponding words “comprise”, “comprised” and “comprises” where they appear.

In addition, the foregoing describes only some embodiments of the invention(s), and alterations, modifications, additions and/or changes can be made thereto without departing from the scope and spirit of the disclosed embodiments, the embodiments being illustrative and not restrictive.

Furthermore, invention(s) have been described in connection with what are presently considered to be the most practical and preferred embodiments, it is to be understood that the invention is not to be limited to the disclosed embodiments, but on the contrary, is intended to cover various modifications and equivalent arrangements included within the spirit and scope of the invention(s). Also, the various embodiments described above may be implemented in conjunction with other embodiments, e.g., aspects of one embodiment may be combined with aspects of another embodiment to realize yet other embodiments. Further, each independent feature or component of any given assembly may constitute an additional embodiment.

What is claimed is:

1. A method of remotely monitoring electromyography therapy, comprising:
receiving an analog signal from an electromyography sensor associated with contraction of a muscle of a first patient;
converting the analog signal to a digital signal;
communicating wirelessly the digital signal to a remote computing device using a communications network;
converting the digital signal into human understandable data by the remote computing device; and
displaying graphically the human understandable data by the remote computing device.

2. The method of claim 1, further comprising:
receiving a second analog signal from a second electromyography sensor associated with contraction of a muscle of a second patient;
converting the second analog signal to a second digital signal;
communicating wirelessly the second digital signal to the remote computing device using the communication network;
converting the second digital signal into human understandable data by the remote computing device; and
displaying graphically the human understandable data associated with the second digital signal by the remote computing device.

3. The method of claim 2 wherein the communications network is the Internet.

4. The method of claim 3 wherein the human understandable data associates the muscle contraction with either the first patient or the second patient.

5. The method of claim 1 wherein communicating wirelessly the digital signal to the remote computing device comprises communicating the digital signal by the electromyography sensor to a mobile device proximate the first patient, then the mobile device communicating a corresponding digital signal to the remote computing device using the communications network.

6. The method of claim 1 further comprising communicating wirelessly the digital signal by the electromyography sensor to a mobile device proximate the first patient using a Bluetooth communication protocol.

7. The method of claim 1 wherein communicating wirelessly the digital signal to the remote computing device comprises communicating the digital signal to a cloud server using the communications network.

8. A computer implemented system for remotely monitoring electromyography therapy, comprising:
a first electromyography sensor configured to be coupled to a first patient and operable to wirelessly transmit a signal associated with contraction of a muscle of the first patient;
a second electromyography sensor configured to be coupled to a second patient and operable to transmit a signal associated with contraction of a muscle of the second patient;
a software program when executed on a computing device remote from the first and second patient operable to:
convert the signals associated with the first patient and the signals associated with the second patient into human understandable data; and
display the human understandable data associated with the first patient and the human understandable data associated with the second patient.

9. The system of claim 8 wherein the display of the human understandable data associated with both the first and second patient is on the same screen.

10. The system of claim 8 wherein the first and second electromyography sensors are operable to transmit the signals using a Bluetooth communication protocol.

11. The system of claim 8 wherein the software program is further operable to send an alert if the human understandable data associated with either the first patient or the second patient indicates a therapy protocol adherence below a predetermined threshold.

12. The system of claim 8 wherein the displayed human understandable data associated with the first patient indicates a repetition of a contraction of the muscle of the first patient and the displayed human understandable data associated with the second patient indicates a repetition contraction of the muscle of the second patient.

13. The system of claim 12 wherein the displayed human understandable data associated with the first patient indicates a strength of a contraction of the muscle of the first patient and the displayed human understandable data associated with the second patient indicates a strength of a contraction of the muscle of the second patient.

14. The system of claim 13 wherein the displayed human understandable data associated with the first patient indicates a date of a contraction of the muscle of the first patient and the displayed human understandable data associated with the second patient indicates a date of a contraction of the muscle of the second patient.

15. The system of claim 14 wherein the displayed human understandable data associated with the first patient indicates an adherence to a therapy protocol of the first patient and the displayed human understandable data associated with the second patient indicates an adherence to a therapy protocol of the second patient.

16. The system of claim 15 wherein the adherence is determined by comparing a number of muscle contraction repetitions to a predetermined goal number of muscle contraction repetitions.

17. The system of claim 8 wherein the human understandable data associated with both the first and second patient is stored in a database.

18. A method of remotely monitoring electromyography therapy of multiple patients, comprising:
communicating using a short range wireless communication protocol a signal from electromyography sensors associated with contraction of a muscle of a plurality of patients;
receiving by respective mobile devices the signals associated with each patient;
communicating wirelessly by each mobile device signals corresponding to the received short range wireless signals to a remote computing device using the Internet;
converting the received signal into human understandable data by the remote computing device; and
displaying the human understandable data by the remote computing device.

19. The method of claim 18 wherein displaying graphically the human understandable data by the remote computing device comprises displaying separately the muscle contraction data of each patient.

20. The method of claim 18 wherein communicating wirelessly by each mobile device signals corresponding to the received short range wireless signals to the remote computing device using the Internet comprises communicating the signals by each mobile device to a cloud server.
